(19) United States
(12) Patent Application Publication
(10) Pub. No.: US 2005/0010196 A1
(43) Pub. Date: Jan. 13, 2005

(54) Osmotic delivery system and method for decreasing start-up times for osmotic delivery systems

(76) Inventors: Pamela J. Fereira, Santa Clara, CA (US); Scott D. Lautenbach, San Mateo, CA (US); Stephen A. Berry, Hollister, CA (US)

Correspondence Address: TRASK BRITT, P.O. BOX 2550, SALT LAKE CITY, UT, 84110, US

(21) Appl. No.: 10/815,169

(22) Filed: Mar. 31, 2004

Related U.S. Application Data
(60) Provisional application No. 60/459,473, filed on Mar. 31, 2003.

(51) Int. Cl. A61K 9/22

(52) U.S. Cl. 604/892.1; 424/422

(57) ABSTRACT

The present invention includes devices and methods for reducing the start-up time of osmotically driven drug delivery systems capable of delivering a desired drug at a controlled rate over time. In particular, the present invention includes osmotic pumps that have a preloaded membrane, which includes a semipermeable material that has been preloaded with a nonaqueous, incompressible liquid filler that is miscible with water. The present invention further includes methods for making such osmotic pumps. The preloaded membranes included in the osmotic pumps according to the present invention have proven to provide significant decreases in average start-up times relative to osmotic pumps that include semipermeable membranes that are not preloaded.

### CROSS-REFERENCE TO RELATED APPLICATION

Pursuant to the provisions of 35 U.S.C. § 119(e), this application claims the benefit of the filing date of provisional patent application Ser. No. 60/459,473, filed Mar. 31, 2003, for “Osmotic Delivery System and Method for Decreasing Start-Up Times for Osmotic Delivery Systems.”

### FIELD OF THE INVENTION

The present invention relates to a delivery device providing delivery of drug at a controlled rate. More particularly, the present invention is directed to drug delivery devices and methods for reducing the start-up time of osmotically driven drug delivery systems capable of delivering a desired drug at a controlled rate over time.

### BACKGROUND

Controlled delivery of beneficial agents, such as drugs, over time has been accomplished by a variety of methods. One method for controlled drug delivery over time involves the use of osmotic delivery devices. These devices can be implanted to release a chosen drug in a controlled manner over a preselected administration period. U.S. Pat. Nos. 3,797,492, 3,987,790, 4,008,719, 4,865,845, 5,057,318, 5,059,423, 5,112,614, 5,137,727, 5,151,093, 5,234,692, 5,234,693, 5,279,608, 5,336,057, 5,728,396, 5,985,305, 5,997,527, 5,997,902, 6,113,938, 6,132,420, 6,217,906, 6,261,584, 6,270,787, and 6,375,978 assigned to ALZA Corporation of Mountain View, Calif., describe various exemplary osmotic delivery devices that may be implanted in a human or animal subject.

Osmotic delivery devices are commonly referred to as “osmotic pumps” and typically include a reservoir, an expandable osmotic material, a drug formulation, and at least one delivery orifice. Where the expandable osmotic material and the drug formulation are formed of separate materials, the expandable osmotic material and the drug formulation may be separated by a member, such as a piston, which is movable within the reservoir. At least a portion of the reservoir included in an osmotic pump is generally semipermeable, allowing water to be taken into the system while working to prevent or minimize the undesired escape of materials forming the expandable osmotic material or the drug formulation from the reservoir. The osmotic material included in an osmotic pump typically draws water from the environment of operation into the osmotic pump through the semipermeable portion of the reservoir. As water is drawn into the device, and in particular into the osmotic material, the osmotic material expands and the drug formulation is discharged through the delivery orifice of the osmotic pump at a chosen release rate or release rate profile.

Though they have proven useful for providing drug delivery at controlled rates, known osmotic pumps also exhibit potential drawbacks. For example, osmotic pumps according to known designs may exhibit start-up times that are inconveniently long. As used herein, the term “start-up time” refers to the time required for an osmotic pump to achieve at least about 90% of the intended release rate or release rate profile after introduction to an environment of operation. The term “environment of operation” refers to any environment into which an osmotic pump can be introduced and is capable of supporting operation of the osmotic pump over a desired period of time. In some instances, the start-up time required for known osmotic pumps extends to two weeks, or more.

In order to reduce the start-up time required by osmotic pumps, ALZA Corporation developed the device and methods described in U.S. Pat. No. 6,132,420 (“the '420 patent”). Though the technology described in the '420 patent provides a reduction in start-up time, the average start-up time exhibited by devices designed according to:the teachings and methods of the '420 patent may still be inconveniently long in certain applications, and a device or method that provides an osmotic pump exhibiting an even further reduction in start-up time would be advantageous. Therefore, it would be an improvement in the art to provide an osmotic pump that both provides a further reduction in average start-up time and works to reduce the potential for a burst release of drug formulation as operation of the device commences.

### SUMMARY OF THE INVENTION

The devices and methods of the present invention address the potential drawbacks of known osmotic delivery devices by providing osmotic pumps that exhibit reduced average start-up times. An osmotic pump according to the present invention includes a preloaded membrane that includes a semipermeable material that has been preloaded with a nonaqueous, incompressible liquid filler that is miscible with water. As they are used herein, the terms “preload” and “preloaded” indicate that a desired amount of liquid filler is absorbed into a semipermeable material included in the preloaded membrane before the osmotic pump with which the preloaded membrane is associated is delivered to an environment of operation. The liquid filler includes a substantially incompressible, nonaqueous liquid that is miscible with water and is absorbable by the material that forms the semipermeable membrane. The amount of liquid filler absorbed into a preloaded membrane included in an osmotic pump of the present invention may vary according to the nature of the material forming the semipermeable membrane, the type of liquid filler used, and the desired reduction in start-up time. It has been found that providing an osmotic pump with a preloaded membrane according to the present invention can result in significant decreases in the average start-up time of the osmotic pump.

In addition to a preloaded membrane, an osmotic pump according to the present invention also includes a reservoir, an osmotic composition, a drug formulation, a delivery orifice, and, optionally, a piston and a second filler distributed around the osmotic composition. The osmotic pump is configured such that water from the chosen environment of operation is drawn through the preloaded membrane into the osmotic composition, causing the expulsion of drug formulation from the delivery orifice. Where desired, a piston may be positioned between the osmotic composition and the drug formulation. The inclusion of a piston can serve to seal the drug formulation from contact with the osmotic composition and may further serve to facilitate more efficient or complete delivery of drug formulation from the osmotic pump. Moreover, depending on the type of osmotic composition used, an osmotic pump of the present invention may be manufactured with a second filler material distributed around the osmotic composition such that any spaces formed between the osmotic composition and the reservoir or, where included, the piston, are occupied by the second filler. Providing an osmotic pump with a second filler distributed around the osmotic composition works to reduce or alleviate undesirable performance characteristics that can result from pockets of air or gas formed around the osmotic composition.

In one embodiment, an osmotic pump of the present invention includes a preloaded membrane that is saturated with liquid filler. A saturated, preloaded membrane according to the present invention includes a semipermeable material and an amount of liquid filler absorbed into the semipermeable material, with the amount of liquid filler absorbed into the semipermeable material resulting in a preloaded membrane that is unable to readily absorb additional amounts of the liquid filler.

In another embodiment, an osmotic device of the present invention includes a semipermeable membrane that is preloaded with sufficient liquid filler to provide an osmotic pump having an average start-up time that is less than 3% of the desired duration of drug delivery. Thus, for example, if an osmotic pump according to this embodiment is designed to deliver a desired drug for a period of about 100 days, the semipermeable membrane will be preloaded with sufficient liquid filler to provide an average start-up time of less than about 3 days.

In yet another embodiment, an osmotic device of the present invention includes an osmotic pump that includes a second filler distributed around the osmotic composition and has a preloaded membrane according to the present invention. The preloaded membrane included in this embodiment of the osmotic pump of the present invention has sufficient liquid filler absorbed therein to provide an osmotic pump exhibiting an average start-up time that is at least 10% shorter than the start-up time provided by an osmotic pump that includes only the second filler.

In another aspect, the present invention includes a method for reducing the start-up time of osmotic pumps. In one embodiment, the method of the present invention includes providing an osmotic pump with a preloaded membrane. Providing the preloaded membrane can be done using various techniques. For instance, a preloaded membrane may be provided simply by exposing a suitable semipermeable material to conditions that result in the absorption of a desired amount of liquid filler. Such conditions may include, for example, immersion of at least a portion of the semipermeable material in an amount of liquid filler under conditions that allow absorption of the desired amount of liquid filler into the semipermeable material within a desired amount of time. However, the method of the present invention may also include any other technique that allows the semipermeable material included in a preloaded membrane to absorb liquid filler in an amount that provides a desired reduction in start-up time.

The method of the present invention may be varied to provide preloaded membranes that include relatively more or less liquid filler. In one embodiment, the method of the present invention includes providing a preloaded membrane that includes a semipermeable material that is saturated with a liquid filler. In another embodiment, the method of the present invention includes providing a preloaded membrane that includes a semipermeable material having an amount of liquid filler absorbed therein that is sufficient to provide a preloaded membrane that provides an average start-up time that is less than 3% of the desired duration of drug delivery.

The method of the present invention may also be varied according to the desired configuration of the osmotic pump. For example, where the method of the present invention includes providing a preloaded membrane for an osmotic pump that includes a second filler distributed around an osmotic composition, the method preferably includes exposing the semipermeable material included in the preloaded membrane to conditions that allow absorption of sufficient liquid filler to create a preloaded membrane that reduces the average start-up time of the osmotic pump by at least 10% relative to an osmotic pump that includes only the second filler. Also, depending on the configuration of the osmotic pump, the method of the present invention preferably includes preloading the semipermeable material with the desired amount of liquid filler after the semipermeable material has been associated with the reservoir of the osmotic pump. In particular, where the semipermeable material included in the preloaded membrane is inserted into or contained within the reservoir of the osmotic pump, the method of the present invention preferably includes preloading the semipermeable material with liquid filler after the semipermeable material has been positioned on, or within, the reservoir.

### BRIEF DESCRIPTION OF THE DRAWINGS

The present invention is described with reference to the accompanying drawings in which like elements bear like reference numerals, and wherein:

FIG. 1 provides a cross-sectional view of an osmotic pump according to the present invention.

FIG. 2 and FIG. 3 provide graphs illustrating the improved release rate performance of osmotic pumps according to the present invention.

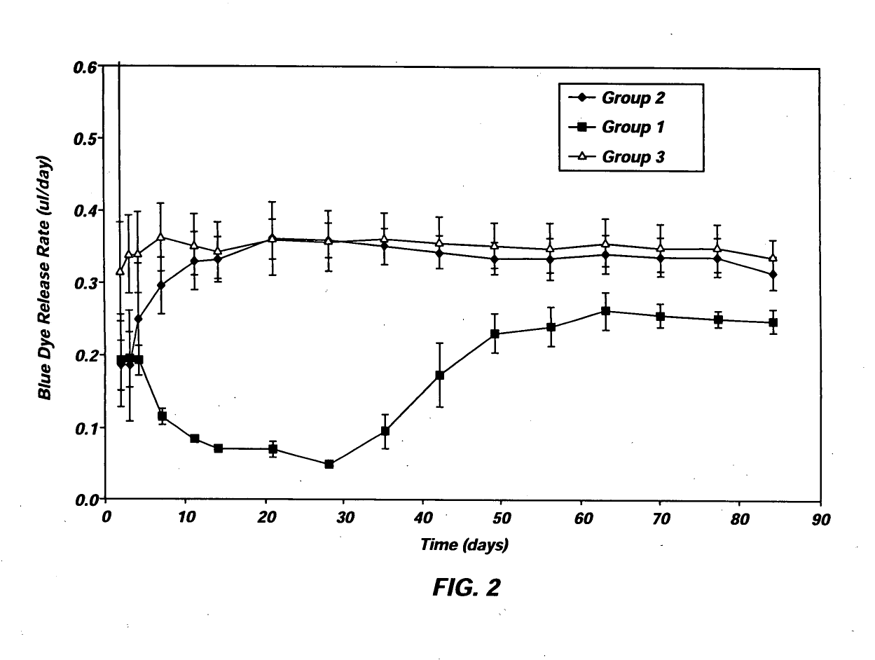

TABLE 1 provides average release rate data for three groups of osmotic pumps, with the osmotic pumps of Group 3 being manufactured according to the present invention and the osmotic pumps included in Group 1 and Group 2 representing osmotic pumps that were not manufactured according to the present invention. The information provided in Table 2 was used to generate the graph illustrated in FIG. 2.

TABLE 2 provides comparative data describing the average start-up time of a group of osmotic pumps manufactured according to the present invention (Group 3) relative to the average start-up times of two groups of osmotic pumps that were not manufactured according to the present invention (Group 1 and Group 2).

### DETAILED DESCRIPTION OF THE INVENTION

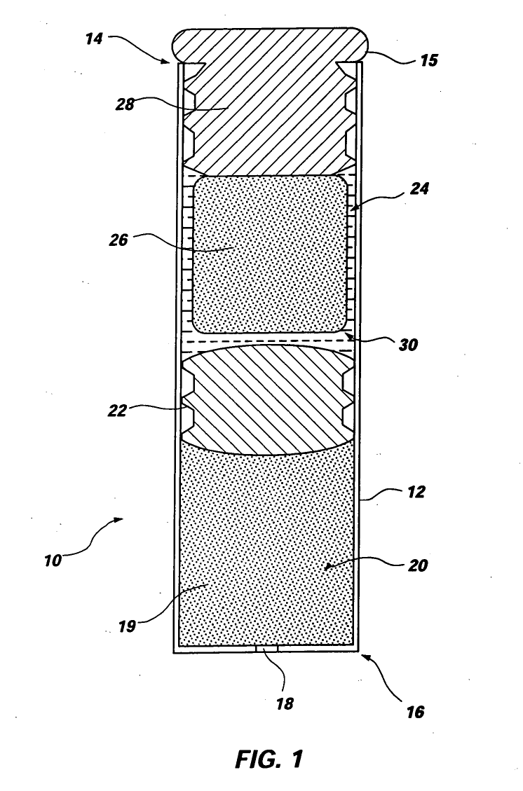

An osmotic pump 10 according to the present invention is illustrated in FIG. 1. The configuration illustrated in FIG. 1 provides only one example of an osmotic pump according to the present invention and is not to be construed as limiting the present invention. The present invention is generally applicable to osmotic pumps, and an osmotic pump according to the present invention may be designed to conform to a wide range of desired sizes or shapes. Moreover, an osmotic pump according to the present invention may be designed for application in various environments or administration by various routes, such as by oral administration, ruminal administration, or implantation.

An osmotic pump 10 according to the present invention includes a preloaded membrane 28 that is affixed to, positioned around, inserted into, or contained within a reservoir 12. An osmotic pump 10 of the present invention also includes an osmotic composition 26, a drug formulation 19, a delivery orifice 18, and optionally, a piston 22 or a second filler 30. Where they are included, a piston 22 is positioned between the osmotic composition 26 and the drug formulation 19, and the second filler 30 is distributed around the osmotic composition 26. It has been found that a significant decrease in start-up time can be achieved by providing an osmotic pump with a preloaded membrane according to the present invention.

The preloaded membrane 28 included in an osmotic pump 10 according to the present invention allows aqueous fluid from the environment of use to pass into the osmotic pump 10 and into the osmotic composition 26. However, the semipermeable material forming the preloaded membrane 28 is largely impermeable to the materials contained within the reservoir as well as other constituents present in the environment of operation. As is shown in FIG. 1, the preloaded membrane 28 of an osmotic pump 10 of the present invention may be formed as a plug that is positioned within a first opening 15 formed in the reservoir 12. Both the preloaded membrane 28 and the reservoir 12 are formed such that the preloaded membrane 28 substantially remains in place during delivery of the drug formulation 19 to an environment of operation. Suitable materials and methods for forming the semipermeable material used in a preloaded membrane 28 included in an osmotic pump 10 of the present invention are detailed in, for example, U.S. Pat. Nos. 3,797,492, 3,987,790, 4,008,719, 4,865,845, 5,057,318, 5,059,423, 5,112,614, 5,137,727, 5,151,093, 5,234,692, 5,234,693, 5,279,608, 5,336,057, 5,728,396, 5,985,305, 5,997,527, 5,997,902, 6,113,938, 6,132,420, 6,217,906, 6,261,584, 6,270,787, and 6,375,978, the contents of which are herein incorporated in their entirety by this reference.

Materials suitable for use as liquid fillers for creating the preloaded membrane 28 of an osmotic pump 10 according to the present invention include any nonaqueous liquid that is substantially incompressible, miscible with water, can be absorbed by the semipermeable material included in the preloaded membrane 28, is suitable for introduction into the intended environment of operation, and is compatible with the other components of the osmotic pump 10. As it is used herein, the term “compatible with” indicates that the liquid does not interact with the components of the osmotic pump (i.e., the reservoir 12, the semipermeable material of the preloaded membrane 28, the osmotic composition 26, the drug formulation 19, the delivery orifice 18, or, where included, the piston 22) in a way that has a substantial adverse affect on the desired performance of the osmotic pump 10. Specific examples of liquid fillers that may be used to create a preloaded membrane 28 of an osmotic pump 10 according to the present invention include, but are not limited to, polyethylene glycols, such as polyethylene glycol (PEG) 400, PEG 1000, propylene glycol (PG), dimethyl sulfoxide (DMSO), and organic liquids. If desired, two or more different liquid filler materials may be used to provide the preloaded membrane 28 of an osmotic pump 10 of the present invention.

Preloading of a semipermeable material to provide a preloaded membrane useful in an osmotic pump of the present invention generally results in swelling of the semipermeable material. Without being limited to a particular mechanism, it is believed that the swelling caused by preloading the liquid filler works to provide the reduction in start-up times exhibited by osmotic pumps according to the present invention. In particular, where an osmotic pump includes a membrane that is not preloaded, water taken into the membrane as the osmotic pump begins to function in an environment of operation does not simply pass through the membrane. Instead, at least some portion of the water entering the membrane as the pump begins operation works to swell the membrane material, and it is believed that as water works to swell the membrane, it is not immediately available to be drawn into the osmotic material and drive the pump. Thus, until the membrane material is swollen, at least to some extent, the osmotic pump will not achieve start-up. However, preloading the membrane with a liquid filler works to swell the semipermeable material included in the membrane before the osmotic pump is introduced into an environment of operation. As a result, it is believed that an osmotic pump including a preloaded membrane according to the present invention will more rapidly achieve a desired flux of water through the membrane because the liquid filler has preswollen the semipermeable material and, in effect, frees water to move through the semipermeable material and into the osmotic composition used to drive the osmotic pump. Over time, the liquid filler initially loaded in the semipermeable material likely elutes out and may be largely replaced by water, but such a process is thought to occur relatively gradually, and does not have a substantial affect on delivery rates.

The amount of liquid filler absorbed into the semipermeable material of the preloaded membrane and osmotic pump of the present invention may vary according to the liquid filler used, the semipermeable material included in the preloaded membrane, and the desired reduction in start-up time. Generally, it is preferred that the preloaded membrane be saturated with the liquid filler, as it is believed that saturation of the semipermeable material included in the preloaded membrane will generally provide the greatest reduction in start-up time. A saturated, preloaded membrane according to the present invention includes semipermeable material and an amount of liquid filler absorbed into the semipermeable material, with the amount of liquid filler absorbed into the semipermeable material resulting in a preloaded membrane that is unable to readily absorb additional amounts of the liquid filler.

The amount of liquid filler required to saturate the membrane forming material will depend on, among other factors, the semipermeable material, the liquid filler, the configuration of the membrane, and the manner in which the membrane is associated with the remaining components of the osmotic pump. Nevertheless, fabrication of a saturated, preloaded membrane according to the present invention is readily achieved by exposing a suitable semipermeable material to a liquid filler under conditions that result in saturation of the semipermeable material by the liquid filler. The conditions required to achieve saturation of a given membrane forming material by a chosen liquid filler can be readily ascertained through routine experimentation by one of ordinary skill in the art.

However, it may not be necessary to saturate the semipermeable material of a preloaded membrane in order to achieve a desired reduction in start-up time, and the preloaded membrane included in an osmotic pump of the present invention is not limited to a semipermeable material that is saturated with liquid filler. In another embodiment of the osmotic pump of the present invention, the preloaded membrane is formed by a semipermeable material that incorporates sufficient liquid filler to provide an osmotic pump exhibiting an average start-up time that is less than 3% of the desired duration of drug delivery. Preferably, the preloaded membrane of such an osmotic pump includes an amount of liquid filler sufficient to provide a start-up time that is less than 2% of the desired duration of drug delivery, with a preloaded membrane including sufficient liquid filler to provide a start-up time that is less than 1% of the desired duration of drug delivery being most preferred.

In addition to a preloaded membrane, an osmotic pump according to the present invention may also include a second additive or filler distributed around the osmotic composition 26. This second filler 30 may be any flowable composition, such as a liquid or gel composition, which is substantially incompressible, is suitable for use in the intended environment of operation, is compatible with the other components of the osmotic pump, works to displace air or gas from around the osmotic composition 26, and does not cause the osmotic composition 26 to swell and freeze-up, as described in U.S. Pat. No. 6,132,420. Materials and methods suitable for providing a second filler 30 suitable for use in an osmotic pump according to the present invention are also described in U.S. Pat. No. 6,132,420, the contents of which are herein incorporated in their entirety by reference. The second filler 30 used in an osmotic pump 10 according to the present invention may be the same as, or different from, the substantially incompressible, nonaqueous liquid filler used to create the preloaded membrane 28 of the osmotic pump 10.

Inclusion of a second filler 30 is particularly helpful where the osmotic composition 26 is formed as a tableted composition. Machining and tableting tolerances require that there be a gap between the osmotic composition 26 and the surrounding reservoir wall. Small irregularities in the shape or contour of the tableted material may also create a gap between the osmotic composition 26 and a piston 22 included in an osmotic pump 10 according to the invention. Such gaps, which can typically range from between about 0.001 to 0.1 inches, are filled with air or other gaseous material, and even the smallest of such air gaps can create a start-up delay of several days to weeks. Additionally, air filled gaps problematically affect the delivery rate of drug formulation when the osmotic pump is subjected to different external pressures, such as when a patient with an implanted osmotic pump scuba dives or travels to higher altitudes. The inclusion of a second filler 30 serves to reduce or eliminate the extent to which any gaps around the osmotic composition 26 are filled with air or another gaseous material and, thereby, works to reduce or eliminate the delays and drug delivery inconsistencies that such gaps can produce.

Where an osmotic pump according to the present invention includes a second filler, the preloaded membrane included in the osmotic pump is not limited to a semipermeable material that is saturated with a liquid filler or to a semipermeable material that includes an amount of liquid filler sufficient to reduce start-up time to less than 3% of the desired duration of drug delivery. In particular, where an osmotic pump according to the present invention includes a second filler distributed around the osmotic composition, the preloaded membrane includes a semipermeable material having an amount of liquid filler absorbed therein that is sufficient to provide a significant decrease in start-up time relative to an osmotic pump that only includes a second filler distributed around the osmotic composition. As it is used herein, the term “significant decrease” indicates a decrease of at least 10%. Where an osmotic pump includes a second filler, the preloaded membrane preferably includes an amount of liquid filler sufficient to decrease the start-up time by at least 25% relative to an osmotic pump that only includes a second filler distributed around the osmotic composition, with a preloaded membrane including an amount of liquid filler sufficient to decrease the start-up time by at least 50% relative to an osmotic pump that only includes a second filler, being especially preferred.

As can be seen by reference to FIG. 1, the reservoir 12 included in an osmotic pump according to the present invention may be elongated and cylindrical having a first end 14 and a second end 16. In the embodiment illustrated in FIG. 1, the first end 14 of the reservoir includes a first opening 15 within which a semipermeable preloaded membrane 28 is positioned. Materials suitable for forming the reservoir 12 must be sufficiently strong to ensure that the reservoir 12 does not leak, crack, break, or significantly distort under stresses to which it is subjected during administration and operation of the osmotic pump 10. In particular, the reservoir 12 is formed of a material that is sufficiently rigid to withstand expansion of the expandable osmotic composition 26 without undergoing substantial changes to the size or shape of the reservoir 12. The material used to form the reservoir 12 is also chosen to be largely impermeable to fluids from the environment of operation and to the material constituents included in the drug formulation 19 and the expandable osmotic composition 26. As it is used herein, the term “largely impermeable” indicates that the migration of materials into or out of the osmotic pump through the material forming the reservoir 12 is so low that any such migration of materials has substantially no adverse impact on the function of the device.

Where the implant is designed for administration to human or animal subjects, the reservoir 12 is preferably formed of chemically inert and biocompatible, natural or synthetic materials known in the art. The reservoir material of an osmotic pump designed for human or animal administration may be formed from a nonbioerodible material, which will remain substantially intact after the functional life of the device. Such a design facilitates recovery or passage of the device after the drug formulation contained therein has been delivered to the subject. However, an osmotic pump 10 of the present invention designed for human or animal administration may also include a reservoir, 12 formed of a bioerodible material that erodes in the environment of operation such that recovery of the reservoir 12 may not be necessary, but the osmotic pump maintains its structural integrity until a desired dose of drug formulation is delivered.

Typical materials suitable for the construction of the reservoir 12 of an osmotic pump 10 according to the present invention include nonreactive polymers and biocompatible metals and alloys. Specific examples of suitable polymers include, but are not limited to, polyimide, polysulfone, polycarbonate, polyethylene, polypropylene, polyvinylchloride-acrylic copolymer, polycarbonate-acrylonitrile-butadiene-styrene, polystyrene, acrylonitrile polymers, such as acrylonitrile-butadiene-styrene terpolymer and the like, halogenated polymers, such as polytetrafluoroethylene, polychlorotrifluorethylene copolymer, tetrafluorethylene and hexafluoropropylene. Metallic materials useful in forming the reservoir include stainless steel, titanium, platinum, tantalum, gold, and their alloys, as well as gold-plated ferrous alloys, platinum-plated ferrous alloys, cobalt-chromium alloys, and titanium nitride coated stainless steel.

The osmotic composition 26 included in the osmotic pump 10 of the present invention may be formed of any material that creates sufficient osmotic pressure to draw water into the osmotic composition 26 through the preloaded membrane 28 such that the osmotic composition 26 causes delivery of the drug formulation 19 at a desired rate over a preselected period of time. Preferably, the osmotic composition 26 is formed as a one or more osmotic tablets formed of an initially solid or nonflowable composition. However, the osmotic composition 26 included in an osmotic pump according to the present invention is not limited to a tableted and initially solid or nonflowable composition. The osmotic composition 26 loaded into a reservoir 12 of an osmotic pump 10 according to the present invention may be formed in any suitable shape, texture, density, and consistency. For example, instead of a solid, tableted composition, it is possible that the expandable osmotic material 26 may be loaded into the reservoir 12 as a powdered material.

The osmotic composition 26 includes an osmotic agent. The osmotic agent included in the osmotic composition 26 is a water-attracting agent that serves to draw water into the osmotic pump 10 through the preloaded membrane 28 and drive the flow of drug formulation 19 out from the osmotic pump 10. The osmotic agent included in the osmotic composition 26 may be an osmagent, an osmopolymer, or a mixture of the two. Methods and formulations for providing osmotic compositions that are suitable for use in an osmotic pump according to the present invention are well known. For example, the patent references that are cited and incorporated by reference herein detail methods and materials suitable for forming osmotic compositions that may be used in an osmotic pump according to the present invention.

Materials that fall within the category of osmagent include materials that are nonvolatile, soluble in water, and create an osmotic gradient suitable for driving the influx of water into the osmotic pump 10. Examples of osmagents that may be useful in the osmotic composition 26 of an osmotic pump 10 of the present invention include, but are not limited to, magnesium sulfate, magnesium chloride, sodium sulfate, lithium sulfate, sodium phosphate, potassium phosphate, d-mannitol, sorbitol, inositol, urea, magnesium succinate, tartaric acid, raffinose, and various monosaccharides, oligosaccharides, and polysaccharides, such as sucrose, glucose, lactose, fructose, and dextran, as well as mixtures of any of these various species.

Materials that fall within the category of osmopolymer are hydrophilic polymers that swell upon contact with water. Osmopolymers may be natural (i.e., of plant or animal origin) or synthetic, and examples of osmopolymers are well known in the art. Particular osmopolymers that may be used in the osmotic composition 26 of an osmotic pump 10 of the present invention include, but are not limited to, poly(hydroxy-alkyl methacrylates) with molecular weights of 30,000 to 5,000,000, poly(vinylpyrrolidone) with molecular weights of 10,000 to 360,000, anionic and cationic hydrogels, polyelectrolyte complexes, poly(vinyl alcohol) having low acetate residual, optionally cross linked with glyoxal, formaldehyde or glutaraldehyde and having a degree of polymerization of 200 to 30,000, a mixture of methyl cellulose, cross linked agar and carboxymethylcellulose, a mixture of hydroxypropyl methylcellulose and sodium carboxymethylcellulose, polymers of N-vinyllactams, polyoxyethylene-polyoxypropylene gels, polyoxybutylene-polyethylene block copolymer gels, carob gum, polyacrylic gels, polyester gels, polyurea gels, polyether gels, polyamide gels, polypeptide gels, polyamino acid gels, polycellulosic gels, Carbopol® acidic carboxy polymers having molecular weights of 80,000 to 200,000, Polyox Polyethylene oxide polymers having molecular weights of 16,000 to 5,000,000, starch graft copolymers, and Aqua-Keeps acrylate polymer polysaccharides.

As can be seen by reference to FIG. 1, where an osmotic pump 10 of the present invention includes an elongated reservoir, at least one delivery orifice 18 can be formed at the second end 16 of the reservoir 12. Where the delivery orifice 18 included in an osmotic pump 10 of the present invention simply includes an orifice formed through the second end 16 of the reservoir 12, such a delivery orifice 18 can be formed using, for example, known molding methods or known mechanical or laser drilling methods. If desired, the reservoir of an osmotic pump of the present invention may include more than one delivery orifice 18. In an alternative embodiment, the delivery orifice 18 of an osmotic pump 10 of the present invention may be formed by an outlet plug (not illustrated) that is positioned at least partially within the reservoir 12. Such an outlet plug may be configured, for example, to provide a delivery orifice that optimizes flow of drug formulation 19 or to regulate back diffusion of environmental fluids into the osmotic pump 10. Outlet plugs suitable for application in an osmotic pump 10 according to the present invention are known in the art and are described in, for example, U.S. Pat. Nos. 5,985,305, 6,217,906, and 5,997,527, the contents of each of which are herein incorporated in their entirety by reference.

Where included in an osmotic pump 10 of the present invention, a movable piston 22 is configured to fit within the reservoir 12 in a sealed manner that allows the piston to be displaced within the reservoir 12 as water is taken into the osmotic composition 26 and the drug formulation 19 is expelled through the delivery orifice 18. In a preferred embodiment, the drug formulation 19 is contained within a first chamber 20 formed within the reservoir 12 and the osmotic composition 26 is contained within a second chamber 24 formed within the reservoir 12, with the first chamber 20 and second chamber 24 being separated by a movable piston 22.

A piston 22 included in an osmotic pump 10 of the present invention may be in the form of a slidable partition or a stationary but stretchable member, such as a diaphragm, partition, or pad. Moreover, a piston 22 suitable for use in an osmotic pump 10 of the present invention is preferably formed of a material that is impermeable to the osmotic composition and the drug formulation, and may include one or more protrusions, which work to form a seal between the piston 22 and the inner surface of the reservoir 12. Materials suitable for use in a piston 22 included in an osmotic pump 10 of the present invention include metallic materials, such as metal alloys, elastomeric materials, such as the nonreactive polymers already mentioned herein, as well as elastomers in general, such as polyurethanes, polyamides, chlorinated rubbers, styrene-butadiene rubbers, and chloroprene rubbers. Methods and materials for providing pistons that are suitable for use in an osmotic pump of the present invention are well known in the art.

The environments of operation where an osmotic pump of the present invention may find use include physiological environments, such as within animal or human bodies. In particular, osmotic pumps of the present invention may be utilized to deliver a desired drug to humans or household, sport, and farm animals. For the administration of beneficial agents to animals or humans, osmotic pumps of the present invention may be implanted into subcutaneous or intraperitoneal environments, wherein aqueous body fluids are available to activate the osmotic engine. Moreover, osmotic pumps of the present invention may be administered to the rumen of ruminant animals, in which embodiment, the osmotic pumps may further comprise a density element for maintaining the device in the rumen for extended periods of time of up to 120 days or longer. Density elements are well known in the art of drug delivery devices.

Although osmotic pumps according to the present invention are preferably designed for, and administered to, human or animal physiological environments, osmotic pumps according to the present invention are generally applicable for the delivery of beneficial agents to an environment of operation and are not limited in utility to physiological environments. For example, the osmotic pumps according to the present invention may be used in intravenous systems (e.g., attached to an IV pump, and IV bag, or an IV bottle) for delivering beneficial agents to animals or humans, systems for blood oxygenation, kidney dialysis or electrophoresis, systems for delivering, for instance, nutrients or growth regulating compounds to cell cultures, as well as in pools, tanks, reservoirs and the like. Therefore, the osmotic pump of the present invention is applicable to the delivery of beneficial agents in general, and the term “drug,” as it is used herein, refers to any beneficial agent that may be delivered to an environment of operation and includes, but is not limited to, medicaments, vitamins, nutrients, biocides, sterilization agents, food supplements, sex sterilants, fertility inhibitors, and fertility promoters. Specific drugs that may be delivered by osmotic pumps of the present invention are detailed, for example, in U.S. Pat. No. 6,132,420, the contents of which are incorporated herein by this reference. Additional examples of drugs that may be delivered by an osmotic device according to the present invention can be found in the other patent references that are cited and incorporated by reference herein.

The drug included in the drug formulation contained within an osmotic pump of the present invention can be present in a wide variety of chemical and physical forms. At the molecular level, the drug may be present as an uncharged molecule, molecular complex, or pharmaceutically acceptable acid addition or base addition salts, such as hydrochlorides, hydrobromides, sulfate, laurylate, oleate, and salicylate. Salts of metals, amines or organic cations may be used for acidic drug compounds. Derivatives of drugs, such as esters, ethers, and amides can also be used. Moreover, the drug formulation included in an osmotic pump according to the present invention may include more than one drug, resulting in an osmotic pump capable of delivering multiple drugs during its functional lifetime.

The drug formulation included in an osmotic pump according to the present invention may include any formulation suitable for delivering a drug from an osmotic pump according to the present invention. The drug formulation may be formulated as any flowable composition, such as a slurry, a suspension, or a solution, capable of delivering the desired drug to a chosen environment of operation. As desired, the drug formulation included in an osmotic pump according to the present invention may include one or more of various ingredients that work to allow delivery of the drug to the desired environment of operation. In particular, the drug formulation included in an osmotic pump according to the present invention may optionally include preservatives, such as one or more antioxidants or other stabilizing agent, permeation enhancers, or carrier materials that are application appropriate. For example, if the osmotic pump is designed for implantation to a human or animal subject, any carrier, preservative, or permeation enhancer used would be a pharmaceutically acceptable material.

In another aspect, the present invention includes a method for reducing the start-up time of osmotic pumps. In one embodiment, the method of the present invention includes providing an osmotic pump with a preloaded membrane. Providing the preloaded membrane can be done using various techniques. For instance, a preloaded membrane may be provided simply by exposing the semipermeable material included in the preloaded membrane to conditions that result in the absorption of a desired amount of liquid filler. Such conditions may include, for example, immersion of at least a portion of the semipermeable material in an amount of liquid filler under conditions that allow absorption of the desired amount of liquid filler into the semipermeable material within a desired amount of time. However, immersion of the semipermeable material may not be necessary to achieve absorption of a desired amount of liquid filler, and the method of the present invention may also include any other technique known in the art that results in the semipermeable material included in a preloaded membrane having a desired amount of liquid filler absorbed therein.

In a preferred embodiment, the method of the present invention includes providing an osmotic pump with a preloaded membrane that includes a semipermeable material that is saturated with a liquid filler. This embodiment of the method of the present invention is preferred because it is presently thought that a preloaded membrane formed of a semipermeable material that is saturated with liquid filler will provide the greatest reduction in start-up time. As has already been discussed, however, saturation of the semipermeable material with a liquid filler may not be necessary to achieve a preloaded membrane that provides a desired reduction in start-up time. Thus, in another embodiment, the method of the present invention includes providing an osmotic pump with a preloaded membrane that includes a semipermeable material having an amount of liquid filler absorbed therein that is sufficient to provide a preloaded membrane that provides an average start-up time that is less than 3% of the desired duration of drug delivery, with methods providing preloaded membranes that provide average start-up times that are less than 2% or less than 1% of the desired duration of drug delivery being especially preferred.

Absorption of the desired amounts of liquid filler into the semipermeable material included in the preloaded membrane can be carried out using techniques known in the art. Again, for example, a preloaded membrane that includes a semipermeable material that is saturated or less than saturated with a liquid filler may be created by immersion of at least a portion of the semipermeable material in the desired liquid filler under conditions that result in the absorption of the desired amount of liquid filler. Because absorption of the liquid filler by the semipermeable material included in a preloaded membrane is typically a thermodynamic process, with absorption of the liquid filler occurring more rapidly as temperature increases, the conditions under which the liquid filler is absorbed may be altered as desired, to reduce or extend the time required for the semipermeable material to absorb the desired amount of liquid filler.

The method of the present invention may also be varied according to the desired configuration of the osmotic pump. For example, where the method of the present invention includes providing a preloaded membrane for an osmotic pump that includes a second filler distributed around an osmotic composition, the method preferably includes exposing the semipermeable material included in the preloaded membrane to conditions that allow absorption of sufficient liquid filler to create a preloaded membrane that reduces the average start-up time of the osmotic pump at least 10% relative to an osmotic pump that includes only the second filler. In such an embodiment, the method preferably includes exposing the semipermeable material to conditions that allow absorption of sufficient liquid filler to create a preloaded membrane that reduces the average start-up time of the osmotic pump by at least 25% relative to an osmotic pump that includes only the second filler, with conditions allowing absorption of sufficient liquid filler to result in a preloaded membrane that reduces the average start-up time by at least 50% relative to an osmotic pump that includes only the second filler being especially preferred. Moreover, depending on the configuration of the osmotic pump, the method of the present invention preferably includes preloading the semipermeable material with the desired amount of liquid filler after the semipermeable material has been associated with the reservoir of the osmotic pump. As they are used herein, the terms “associate with” or “associated with” indicate that the semipermeable material has been affixed to, positioned around, inserted into, or is contained within a reservoir.

In one embodiment, therefore, the method of the present invention includes first providing a subassembly that includes the semipermeable material used to form the preloaded membrane associated with a reservoir suitable for use in an osmotic pump. The semipermeable material included in the subassembly is then exposed to conditions that cause the absorption of a desired amount of liquid filler to create a preloaded membrane that provides a desired reduction in start-up time. This embodiment of the present invention is particularly preferred where the preloaded membrane included in an osmotic pump of the present invention is inserted into or contained within the reservoir. As is discussed herein, preloading of the semipermeable material with a liquid filler typically causes the semipermeable material to swell, and as a result, preloading of the semipermeable material before it is inserted into or positioned within the reservoir may prevent proper placement of the resulting preloaded membrane.

Where the method of the present invention includes first providing a subassembly including at least the semipermeable material of the preloaded membrane associated with a reservoir, the subassembly may be one of various stages of completion. For example, the subassembly may include only the semipermeable material and the reservoir. Alternatively, where the method includes providing an osmotic pump having a piston and a second filler distributed around the osmotic composition, the subassembly may include not only the reservoir and the semipermeable material, but also the piston, the osmotic composition, and the second filler. Further, the subassembly used in a method according to the present invention may include all aspects of the osmotic pump other than the preloaded membrane. In such an instance, the preloaded membrane would be simply completed by exposing the semipermeable material associated with the subassembly to conditions that result in the absorption of a desired amount of liquid filler.

### EXAMPLE

In order to evaluate the benefits provided by the present invention, three groups of 16 osmotic pumps were manufactured and tested for release rate performance. Each of the osmotic pumps manufactured were loaded with a simulated drug formulation that included 2% blue dye in a carrier formed of 30% PVP and 70% DMSO (to simulate, for example, the typical viscosity of leuprolide acetate/DMSO solutions). In addition, each of the osmotic pumps was manufactured to provide a targeted drug formulation release rate of 0.35 μl/day for at least one year. To measure the release rate performance, each system was placed in a controlled temperature water bath maintained at 37° C., and the rate at which the simulated drug formulation was released from each system was measured using a Shimadzu 1601 UV/Vis Spectrophotometer.

The first group of 16 osmotic pumps were manufactured using the following components:
Reservoir (Titanium, Ti6Al4V alloy) (4 mm outside diameter, 3 mm inside diameter)
Piston (C-Flex®)
Lubricant (silicone medical fluid)
Osmotic Composition (40 mg osmotic engine tablets formed using 76.4% NaCl, 15.5% sodium carboxymethyl cellulose, 6% povidone, 0.5% Mg Stearate, and 1.6% water)
Semipermeable Membrane (polyurethane polymer, injection molded to desired plug shape)
Back Diffusion Regulating Outlet (polyethylene)
Simulated Drug formulation (2% blue dye in a carrier of 30% PVP and 70% DMSO)

Using these components, the first group of osmotic pumps was manufactured by, first, lightly lubricating the piston and inner diameter of the reservoir using the silicon medical fluid. The piston was then inserted ˜0.5 cm into the reservoir at the first end (hereinafter “the membrane end”). Two osmotic engine tablets (40 mg each) were then inserted into the reservoir from the membrane end. After insertion of the osmotic engine tablets, the resulting osmotic composition was flush with the membrane end of the reservoir. A semipermeable membrane plug (hereinafter “the membrane plug” or “plug”) was inserted into the reservoir by lining up the plug with the membrane end of the reservoir and pushing gently until the retaining features of the plug were fully engaged in the reservoir. The simulated drug formulation was loaded into a syringe, which was then used to fill the reservoir from the second end (hereinafter “the outlet end”) by injecting the simulated drug formulation into the reservoir until the formulation was ˜3 mm from the end. The filled reservoir was centrifuged (outlet end “up”) to remove any air bubbles that were trapped in the simulated drug formulation during filling. The back diffusion regulating outlet was screwed into the outlet end of the reservoir until completely engaged. As the outlet was screwed in, excess amount of simulated drug formulation exited out of the delivery orifice, ensuring a uniform fill.

The second group of 16 osmotic pumps, which were used as a control, were manufactured using the same components and methods as were used to manufacture the first group of osmotic pumps, except that the second group of osmotic pumps was manufactured to include PEG 400 as a fluid filler distributed around the osmotic engine tablets that formed the osmotic composition. To accomplish this, 8 mg of PEG 400 was added through the membrane end of the reservoir after insertion of the piston. After the addition of the PEG 400, the two osmotic engine tablets were inserted into the reservoir through the membrane end, resulting in the PEG 400 substantially filling air gaps existing between tablets and the piston or the inner diameter of the reservoir. The piston, PEG 400, and osmotic engine tablets were added to each of the second group of osmotic pumps such that top of the osmotic composition formed by the osmotic engine tablets was flush with the first end of the reservoir. The membrane plug, the simulated drug composition, and the back diffusion-regulating outlet of each of the second group of osmotic pumps were provided according to the methods already described.

The third group of 16 osmotic pumps, which represented exemplary osmotic pumps according to the present invention, was manufactured using the same components and methods as were used to manufacture the second group of osmotic pumps, except that PEG 400 was preloaded into the membrane plug after the plug was inserted into the reservoir. In this instance, the PEG 400 was allowed to absorb into the membrane plugs included in each of the osmotic pumps of the third group under conditions that allowed saturation of the plugs by the PEG 400. Specifically, after insertion of the membrane plugs into the reservoirs of each of the third group of osmotic pumps, the membrane end of the subassembly was positioned in 2 ml plastic vials and PEG 400 was added to the vials until the exposed portion of the membrane plugs was completely immersed in PEG 400. The subassemblies were left in the in the vials for 14 days. Upon removal from the plastic vials, the membrane end of each of the subassemblies was wiped clean.

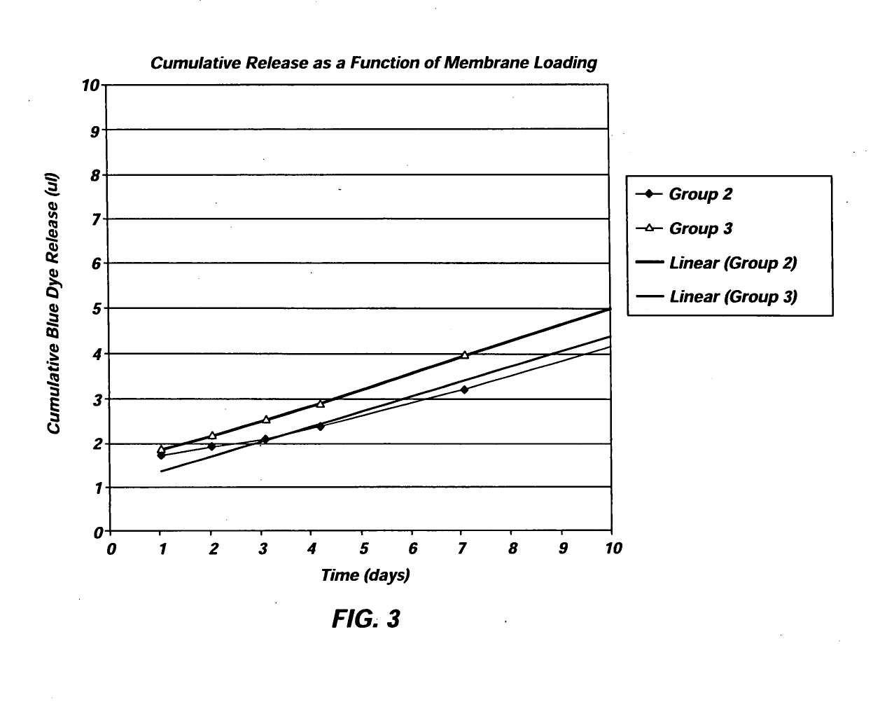

The average release rate performance exhibited by each of the three groups of 16 osmotic pumps is described in Table 1 and illustrated in FIG. 2 and FIG. 3.

TABLE 1
Average Data for FIG. 2

| | 0.35 μl/day | | | |
|---|---|---|---|---|
| Target | Cumulative | Group | | |
| Release = | Time (days) | 2 | Group 1 | Group 3 |
| | 1.04 | 1.64 | 1.75 | 1.79 |
| | 2.04 | 0.19 | 0.19 | 0.32 |
| | 3.11 | 0.18 | 0.19 | 0.34 |
| | 4.18 | 0.25 | 0.19 | 0.34 |
| | 7.08 | 0.29 | 0.11 | 0.36 |
| | 11.18 | 0.33 | 0.08 | 0.35 |
| | 14.13 | 0.33 | 0.07 | 0.34 |
| | 20.88 | 0.36 | 0.07 | 0.36 |
| | 27.97 | 0.36 | 0.05 | 0.36 |
| | 35.10 | 0.35 | 0.09 | 0.36 |
| | 42.00 | 0.34 | 0.17 | 0.36 |
| | 49.04 | 0.33 | 0.23 | 0.35 |
| | 56.03 | 0.33 | 0.24 | 0.35 |
| | 63.00 | 0.34 | 0.26 | 0.36 |
| | 69.98 | 0.34 | 0.26 | 0.35 |
| | 77.17 | 0.34 | 0.25 | 0.35 |
| | 83.98 | 0.32 | 0.25 | 0.34 |

As is easily appreciated by reference to the table and figures, the osmotic pumps prepared according to the present invention provide a significantly reduced average start-up time, allowing the osmotic pump to reach target, or near target, drug delivery rates more quickly than osmotic pumps that do not include a preloaded membrane. Moreover, as can be seen in Table 2, the average start-up time required by the third group of osmotic pumps Was only 0.8% of the desired duration of drug delivery, while the average start-up time of the osmotic pumps of the second group represented 3.0% of the desired duration of drug delivery. Significantly, the osmotic pumps of the first group required more than 50 days to achieve steady state delivery of the simulated drug formulation and never achieved start-up.

TABLE 2
Percent Start-Up to Total Delivery Duration

| | Targeted | | | % Start-Up to |
|---|---|---|---|---|
| | Release Rate | Targeted Release | Average Start-Up | Targeted Delivery |
| Osmotic Pump | (μl/day) | Duration (Days) | Time (days) | Duration |
| Group 1 - No | 0.35 | 365 | Start-up never | |
| Filler/No | | | achieved | |
| Preloaded | | | | |
| Membrane | | | | |
| Group 2 - Filler | 0.35 | 365 | 11 | 3.0 |
| Only | | | | |
| Group 3 - Filler | 0.35 | 365 | 3 | 0.8 |
| and Preloaded | | | | |
| Membrane | | | | |

1. An osmotic delivery device comprising:
a reservoir having at least one drug delivery orifice;
an osmotic composition contained within the reservoir;
a drug formulation contained within the reservoir; and
a preloaded membrane comprising a semipermeable material and a liquid filler material contained within the semipermeable material, wherein the preloaded membrane is configured to allow a fluid to pass from an environment surrounding the reservoir into the osmotic composition.

2. The osmotic delivery device of claim 1, further comprising a slidable partition disposed within the reservoir between the osmotic composition and the drug formulation.

3. The osmotic delivery device of claim 1, further comprising a second filler material distributed around the osmotic composition within the reservoir.

4. The osmotic delivery device of claim 3, wherein the second filler material comprises a composition that is substantially the same as a composition of the liquid filler material of the preloaded membrane.

5. The osmotic delivery device of claim 3, wherein the semipermeable material of the preloaded membrane contains a sufficient amount of the liquid filler material such that the osmotic delivery device is configured to exhibit an average start-up time that is at least 10% less than the start-up time of an osmotic delivery device without a preloaded membrane.

6. The osmotic delivery device of claim 5, wherein the semipermeable material of the preloaded membrane contains a sufficient amount of the liquid filler material such that the osmotic delivery device is configured to exhibit an average start-up time that is at least 25% less than the start-up time of an osmotic delivery device without a preloaded membrane.

7. The osmotic delivery device of claim 6, wherein the semipermeable material of the preloaded membrane contains a sufficient amount of the liquid filler material such that the osmotic delivery device is configured to exhibit an average start-up time that is at least 50% less than the start-up time of an osmotic delivery device without a preloaded membrane.

8. The osmotic delivery device of claim 1, wherein the semipermeable material of the preloaded membrane contains a sufficient amount of the liquid filler material such that the osmotic delivery device is configured to exhibit an average start-up time that is at least less than 3% of a desired duration of drug delivery for the osmotic delivery device.

9. The osmotic delivery device of claim 8, wherein the semipermeable material of the preloaded membrane contains a sufficient amount of the liquid filler material such that the osmotic delivery device is configured to exhibit an average start-up time that is at least less than 2% of a desired duration of drug delivery for the osmotic delivery device.

10. The osmotic delivery device of claim 9, wherein the semipermeable material of the preloaded membrane contains a sufficient amount of the liquid filler material such that the osmotic delivery device is configured to exhibit an average start-up time that is at least less than 1% of a desired duration of drug delivery for the osmotic delivery device.

11. The osmotic delivery device of claim 1, wherein the semipermeable material of the preloaded membrane is saturated with the liquid filler material.

12. The osmotic delivery device of claim 1, wherein the liquid filler material comprises at least one of a polyethylene glycol, a propylene glycol, a dimethyl sulfoxide, and an organic liquid.

13. The osmotic delivery device of claim 1, wherein the liquid filler material comprises at least two different liquid filler materials.

14. The osmotic delivery device of claim 1, wherein the preloaded membrane comprises a plug that is positioned within an opening in the reservoir.

15. An osmotic delivery device comprising:
a reservoir having at least one drug delivery orifice;
an osmotic composition contained within the reservoir;
a drug formulation contained within the reservoir; and
a preloaded membrane comprising a semipermeable material and a liquid filler material contained within the semipermeable material, wherein the preloaded membrane is configured as a plug that is positioned within an opening in the reservoir, and wherein the liquid filler material comprises at least one of a polyethylene glycol, a propylene glycol, a dimethyl sulfoxide, and an organic liquid.

16. A method of reducing the delivery start-up time of an osmotic delivery device comprising:
providing a reservoir having at least one drug delivery orifice;
disposing an osmotic composition within the reservoir;
disposing a drug formulation within the reservoir;
preloading a membrane comprising a semipermeable material with a liquid filler material; and
associating the membrane with the reservoir such that the membrane is configured to allow a fluid to pass from an environment surrounding the reservoir into the osmotic composition.

17. The method of claim 16, further comprising disposing a glidable partition within the reservoir between the osmotic composition and the drug formulation.

18. The method of claim 16, further comprising distributing a second filler material around the osmotic composition within the reservoir.

19. The method of claim 18, further comprising selecting the second filler material to comprise a composition that is substantially the same as a composition of the liquid filler material of the membrane.

20. The method of claim 18, wherein preloading the membrane comprises exposing the semipermeable material of the membrane to conditions that allow adsorption of a sufficient amount of the liquid filler material to reduce the average start-up time of the osmotic delivery device at least 10% relative to an osmotic delivery device wherein the membrane has not been preloaded.

21. The method of claim 20, wherein preloading the membrane comprises exposing the semipermeable material of the membrane to conditions that allow adsorption of a sufficient amount of the liquid filler material to reduce the average start-up time of the osmotic delivery device at least 25% relative to an osmotic delivery device wherein the membrane has not been preloaded.

22. The method of claim 21, wherein preloading the membrane comprises exposing the semipermeable material of the membrane to conditions that allow adsorption of a sufficient amount of the liquid filler material to reduce the average start-up time of the osmotic delivery device at least 50% relative to an osmotic delivery device wherein the membrane has not been preloaded.

23. The method of claim 16, wherein preloading the membrane comprises preloading the semipermeable material of the membrane with an amount of liquid filler material sufficient to provide and average start-up time that is at least less than 3% than a desired duration of drug delivery for the osmotic delivery device.

24. The method of claim 23, wherein preloading the membrane comprises preloading the semipermeable material of the membrane with an amount of liquid filler material sufficient to provide and average start-up time that is at least less than 2% than a desired duration of drug delivery for the osmotic delivery device.

25. The method of claim 24, wherein preloading the membrane comprises preloading the semipermeable material of the membrane with an amount of liquid filler material sufficient to provide and average start-up time that is at least less than 1% than a desired duration of drug delivery for the osmotic delivery device.

26. The method of claim 16, wherein preloading the membrane comprises saturating the semipermeable material of the membrane with the liquid filler material.

27. The method of claim 16, further comprising selecting the liquid filler material to comprise at least one of a polyethylene glycol, a propylene glycol, a dimethyl sulfoxide, and an organic liquid.

28. The method of claim 16, wherein preloading the membrane comprises preloading the membrane with at least two different liquid filler materials.

29. The method of claim 16, wherein associating the membrane with the reservoir comprises inserting the membrane into an opening within the reservoir.

30. The method of claim 16, further comprising preloading the membrane with the liquid filler material prior to associating the membrane with the reservoir.

31. The method of claim 16, wherein preloading the membrane with the liquid filler material comprises immersing at least a portion of the semipermeable material of the membrane in an amount of the liquid filler material.
